(19) United States
(12) Patent Application Publication
(10) Pub. No.: US 2025/0054584 A1
(43) Pub. Date: Feb. 13, 2025

(54) COMPUTER-READABLE RECORDING MEDIUM STORING ACTIVE LEARNING PROGRAM, METHOD, AND APPARATUS

(71) Applicant: Fujitsu Limited, Kawasaki-shi (JP)

(72) Inventor: Yasufumi SAKAI, Fuchu (JP)

(73) Assignee: Fujitsu Limited, Kawasaki-shi (JP)

(21) Appl. No.: 18/755,797

(22) Filed: Jun. 27, 2024

(30) Foreign Application Priority Data
Aug. 10, 2023 (JP) 2023-131606

(51) Int. Cl. G16C 20/70; G16C 20/30

(52) U.S. Cl. CPC G16C 20/70; G16C 20/30

(57) ABSTRACT

A non-transitory computer-readable recording medium stores an active learning program for causing a computer to execute a process including: predicting, based on a second machine learning model trained by using a set of a plurality of pieces of data and a prediction error of a first machine learning model for each of the plurality of pieces of data as training data, a prediction error of the first machine learning model for each of a plurality of pieces of unlabeled data; generating labeled data by assigning a correct answer label to unlabeled data selected from the plurality of pieces of unlabeled data, based on the predicted prediction error; and retraining the first machine learning model by using the generated labeled data.

### CROSS-REFERENCE TO RELATED APPLICATION

This application is based upon and claims the benefit of priority of the prior Japanese Patent Application No. 2023-131606, filed on Aug. 10, 2023, the entire contents of which are incorporated herein by reference.

### FIELD

The embodiments discussed herein are related to a non-transitory computer-readable recording medium storing an active learning program, an active learning method, and an active learning apparatus.

### BACKGROUND

In the related art, a neural network that predicts energy of a molecule based on structure data of the molecule is proposed. The neural network is trained by supervised learning using labeled data consisting of a “structure” and “energy”. For example, the “energy” is calculated by a numerical calculation method called Density Functional Theory (DFT). A calculation time of the energy of a molecule by DFT is very long, and the calculation of the energy of one structure may take half a day to three days. Therefore, it is difficult to collect a large amount of labeled data for the supervised learning.

International Publication Pamphlet No. WO2022/113338, Kristof T.Schutt, Oliver T.Unke, and Michael Gastegger, “Equivariant Message Passing for the Prediction of Tensorial Properties and Molecular Spectra,” PMLR, 2021, Johannes Gasteiger, Muhammed Shuaibi, Anuroop Sriram, Stephan Gunnemann, Zachary Ulissi, C.Lawrence Zitnick, and Abhishek Das, “GemNet-OC: Developing Graph Neural Networks for Large and Diverse Molecular Simulation Datasets,” Transactions on Machine Learning Research, 2022, and Joseph Musielewicz, Xiaoxiao Wang, Tian Tian, and Zachary Ulissi, “FINETUNA: Fine-tuning Accelerated Molecular Simulations,” arXiv: 2205.01223v2 [physics.comp-ph] 1 Jul. 2022 are disclosed as related art.

### SUMMARY

According to an aspect of the embodiments, a non-transitory computer-readable recording medium stores an active learning program for causing a computer to execute a process including: predicting, based on a second machine learning model trained by using a set of a plurality of pieces of data and a prediction error of a first machine learning model for each of the plurality of pieces of data as training data, a prediction error of the first machine learning model for each of a plurality of pieces of unlabeled data; generating labeled data by assigning a correct answer label to unlabeled data selected from the plurality of pieces of unlabeled data, based on the predicted prediction error; and retraining the first machine learning model by using the generated labeled data.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram describing an NN for prediction of molecule energy;

FIG. 2 is a diagram describing the related art;

FIG. 3 is a diagram describing an uncertainty prediction NN in the related art;

FIG. 4 is a functional block diagram of an active learning apparatus according to the present embodiment;

FIG. 5 is a diagram describing an energy prediction model;

FIG. 6 is a diagram describing an error prediction model;

FIG. 7 is a diagram illustrating an example of a relationship between a calculation value of energy by DFT and a prediction value of the energy by the energy prediction model;

FIG. 8 is a block diagram illustrating a schematic configuration of a computer that functions as the active learning apparatus;

FIG. 9 is a flowchart illustrating an example of an active learning process;

FIG. 10 is a diagram illustrating an outline of a process in the active learning apparatus; and

FIG. 11 is a diagram illustrating a result of verifying an effect of the present method.

### DESCRIPTION OF EMBODIMENTS

Thus, an active learning method for increasing accuracy of a neural network with as few labeled data as possible is proposed. For example, there is proposed a technique for predicting uncertainty of a prediction value of energy for unlabeled data by using a machine learning model that predicts the uncertainty of the prediction value of the energy by a neural network. With this technique, unlabeled data having a large predicted uncertainty is labeled, and the labeled data is used to train the neural network that predicts the energy.

As a technique related to active learning, there is proposed an information processing apparatus that receives a training example including feature amounts, assigns a label to the training example, generates one or more student models by using the training example to which the label is assigned, and calculates an error between prediction by the student model and the label. This apparatus generates an error prediction model that is a model for predicting the error, and outputs a use example in which the error is predicted to be increased based on the error prediction model.

Meanwhile, in the related art using the machine learning model that predicts uncertainty of the prediction value of the energy by the neural network, a plurality of neural networks are desirable to predict the uncertainty. For example, in the related art, there is a problem in that the plurality of neural networks are to be prepared for the active learning, and a training cost by the active learning is high.

As one aspect, an object of the disclosed technique is to reduce a training cost by active learning for improving accuracy of a machine learning model with as few labeled data as possible.

Hereinafter, an example of embodiments according to the disclosed technique will be described with reference to the drawings.

Before describing details of the embodiments, problems in active learning directed to supervised learning of a machine learning model for prediction of molecule energy will be described.

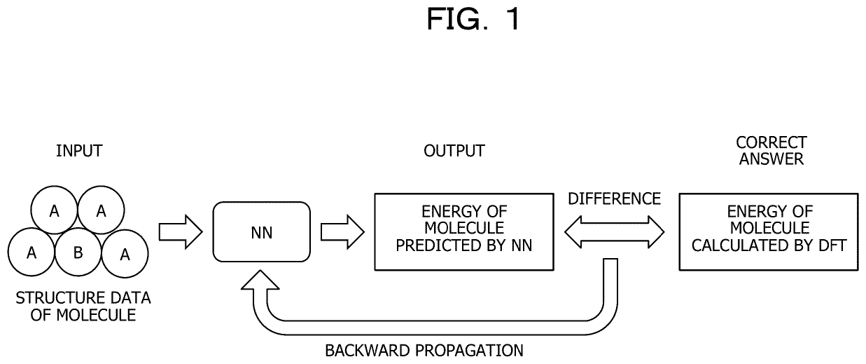

As illustrated in FIG. 1, a neural network (hereafter, also referred to as “NN”) that predicts energy of a molecule based on structure data of the molecule is trained by supervised learning using labeled data including “structure” and “energy”. For example, the structure data of the molecule is input to the NN, and a difference between the predicted energy of the molecule output from the NN and energy of a correct answer is back-propagated to update parameters of the NN. For example, the energy to be the correct answer is calculated by a numerical calculation method called DFT. Since a calculation time of the energy of the molecule by DFT is very long, it is difficult to collect a large amount of labeled data for the supervised learning.

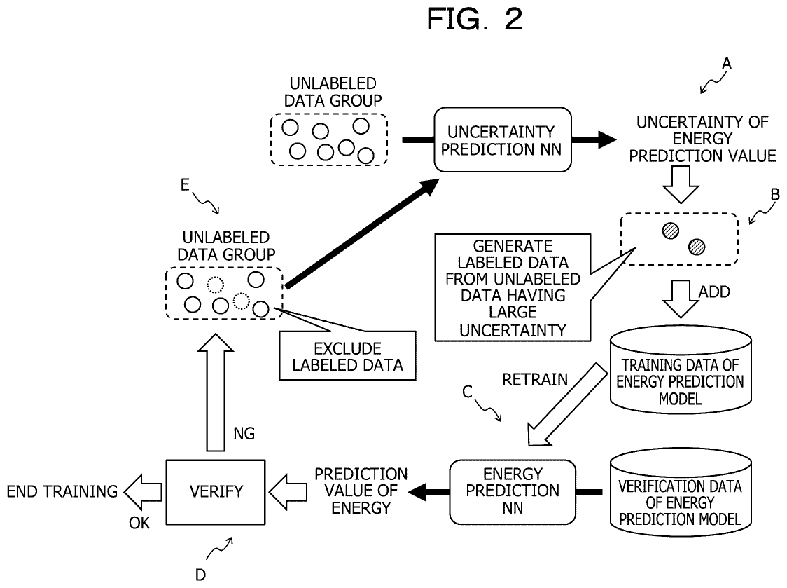

Thus, an active learning method for increasing accuracy of the NN with as few labeled data as possible is proposed. For example, as indicated by A in FIG. 2, an information processing apparatus that executes active learning predicts uncertainty of a prediction value of energy, for each unlabeled data included in an unlabeled data group, by using an uncertainty prediction NN for predicting the uncertainty of the prediction value of the energy. Among a plurality of pieces of unlabeled data, the information processing apparatus selects unlabeled data with large uncertainty predicted by the uncertainty prediction NN. As illustrated in B of FIG. 2, the information processing apparatus generates labeled data by assigning energy calculated by DFT or the like, for example, as a correct answer label to the selected unlabeled data.

As indicated by C in FIG. 2, the information processing apparatus retrains the energy prediction NN for predicting the energy, by using training data to which the generated labeled data is added. As indicated by D in FIG. 2, the information processing apparatus evaluates the retrained energy prediction NN, by using verification data, and ends the training in a case where accuracy of the energy prediction NN satisfies a criterion. By contrast, in a case where the accuracy of the energy prediction NN does not satisfy the criterion, as indicated by E in FIG. 2, the information processing apparatus excludes the data to which the label is assigned in B described above, from the unlabeled data group, and the process is repeated from the process of predicting the uncertainty.

In this manner, by predicting the uncertainty of the prediction value of the energy by the uncertainty prediction NN and selecting unlabeled data having large uncertainty as data to be labeled, data with which the energy prediction NN may be efficiently trained is selected.

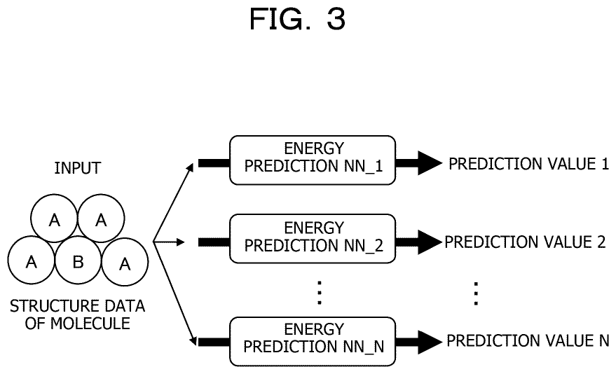

Meanwhile, for example, in the technique described in Joseph Musielewicz, Xiaoxiao Wang, Tian Tian, and Zachary Ulissi, “FINETUNA: Fine-tuning Accelerated Molecular Simulations,” arXiv: 2205.01223v2 [physics.comp-ph] 1 Jul. 2022, this uncertainty prediction NN is configured with a plurality of neural networks as illustrated in FIG. 3. For example, in a plurality of (N in the example of FIG. 3) energy prediction NNs each having a different weight or the like, structure data of the same molecule is input, and energy of the molecule is predicted. A variation in the prediction value of each energy prediction NN is used as uncertainty of the structure data. Due to such a configuration, in the technique described in Joseph Musielewicz, Xiaoxiao Wang, Tian Tian, and Zachary Ulissi, “FINETUNA: Fine-tuning Accelerated Molecular Simulations,” arXiv: 2205.01223v2 [physics.comp-ph] 1 Jul. 2022, it is desirable to prepare the plurality of neural networks in order to predict the uncertainty, and a training cost is increased.

Thus, in the present embodiment, a method of predicting uncertainty of a prediction value of energy with a single neural network is proposed. Hereinafter, an active learning apparatus according to the present embodiment will be described in detail.

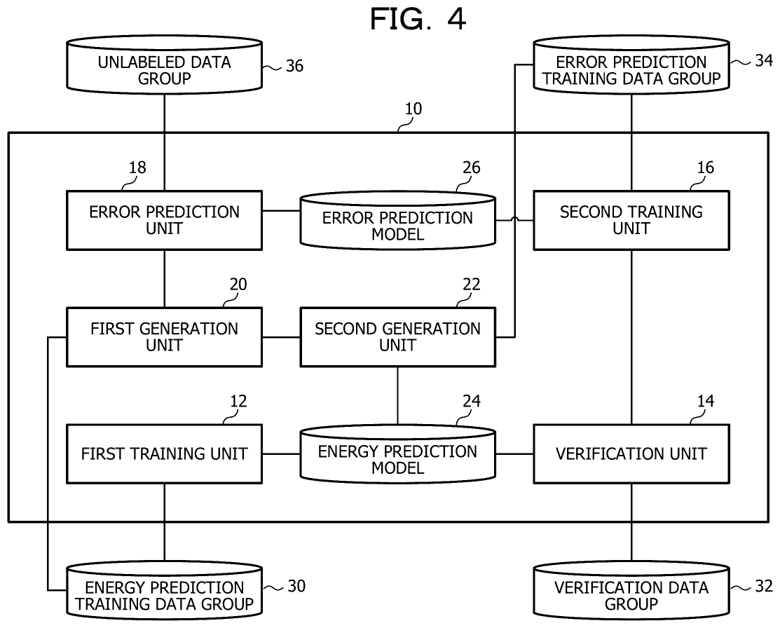

As illustrated in FIG. 4, an active learning apparatus 10 functionally includes a first training unit 12, a verification unit 14, a second training unit 16, an error prediction unit 18, a first generation unit 20, and a second generation unit 22. An energy prediction model 24 and an error prediction model 26 are stored in a predetermined storage region of the active learning apparatus 10. The energy prediction model 24 is an example of a “first machine learning model” of the disclosed technique, and the error prediction model 26 is an example of a “second machine learning model” of the disclosed technique.

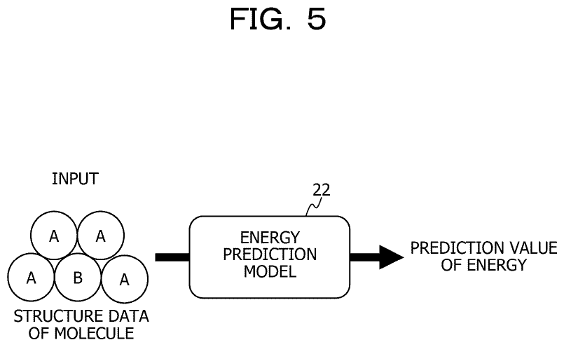

By using an energy prediction training data group 30, the first training unit 12 trains the energy prediction model 24. The energy prediction training data group 30 includes a plurality of pieces of energy prediction training data which is a set of structure data of a molecule and a correct answer label of energy of the molecule. The energy prediction model 24 is a machine learning model configured with a neural network or the like. As illustrated in FIG. 5, in a case where structure data of a molecule is input, the energy prediction model 24 outputs a prediction value of energy of the molecule.

For example, the first training unit 12 updates a parameter of the energy prediction model 24 such that a prediction value of the energy output by inputting the structure data of the molecule of the energy prediction training data to the energy prediction model 24 coincides with the energy indicated by the correct answer label. In a case where labeled data generated by the first generation unit 20, which will be described below, is added to the energy prediction training data group 30, the first training unit 12 retrains the energy prediction model 24 by using the energy prediction training data group 30 after the addition.

By using a verification data group 32, the verification unit 14 verifies accuracy of the energy prediction model 24. The verification data group 32 includes, as verification data, a plurality of pieces of labeled data which are labeled data obtained by assigning a correct answer label of the energy of the molecule to the structure data of the molecule and are not used for training of the energy prediction model 24.

Based on the prediction value predicted by the energy prediction model 24 for the structure data of the molecule of the verification data and the correct answer label of the verification data, the verification unit 14 calculates prediction accuracy of the energy prediction model 24. The verification unit 14 verifies whether or not the prediction accuracy satisfies a predetermined criterion. In a case where the prediction accuracy satisfies the predetermined criterion, the verification unit 14 ends the training of the energy prediction model 24. By contrast, in a case where the prediction accuracy does not satisfy the predetermined criterion, the verification unit 14 repeatedly executes the process of each functional unit, and retrains the energy prediction model 24 and the error prediction model 26.

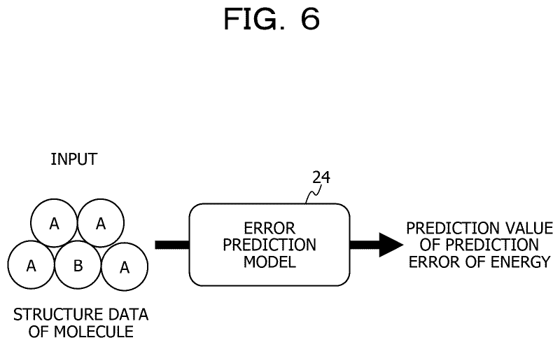

By using an error prediction training data group 34, the second training unit 16 trains the error prediction model 26. A plurality of pieces of error prediction training data, which is a set of the structure data of the molecule and a correct answer label of an error (hereafter, also referred to as a “prediction error”) of the prediction value in a case where the energy of the molecule is predicted by the energy prediction model 24, are included in the error prediction training data group 34. The error prediction model 26 is a machine learning model configured with a single neural network or the like. As illustrated in FIG. 6, in a case where structure data of a molecule is input, the error prediction model 26 outputs a prediction value of a prediction error of energy of the molecule.

For example, the second training unit 16 updates a parameter of the error prediction model 26 such that the prediction value of the prediction error of the energy output by inputting the structure data of the molecule of the error prediction training data to the error prediction model 26 coincides with a prediction error indicated by the correct answer label. In a case where the error prediction training data generated by the second generation unit 22, which will be described below, is added to the error prediction training data group 34, the second training unit 16 retrains the error prediction model 26 by using the error prediction training data group 34 after the addition.

Based on the error prediction model 26, the error prediction unit 18 predicts a prediction error of the energy prediction model 24 for each of a plurality of pieces of unlabeled data included in the unlabeled data group 36.

Based on the prediction error predicted by the error prediction unit 18, the first generation unit 20 selects unlabeled data to be labeled from the plurality of pieces of unlabeled data. For example, in the present embodiment, the prediction error predicted by the error prediction unit 18 is used as uncertainty of the prediction result of the unlabeled data for the energy prediction model 24.

For example, the first generation unit 20 selects unlabeled data with which a prediction error predicted by the error prediction model 26 is equal to or more than a predetermined value or a predetermined number of pieces of unlabeled data in descending order of the prediction errors, among the plurality of pieces of unlabeled data included in the unlabeled data group 36. For example, unlabeled data with large uncertainty is selected. The first generation unit 20 acquires information on energy to be a correct answer label for the selected unlabeled data.

For example, the first generation unit 20 acquires information on energy calculated by DFT, for structure data of a molecule indicated by the unlabeled data. The calculation of the energy by DFT may be executed inside the active learning apparatus 10 or may be executed by an external apparatus. For the execution by the external apparatus, the first generation unit 20 outputs the structure data of the molecule indicated by the unlabeled data to the external apparatus, and acquires a calculation result from the external apparatus. The first generation unit 20 may present the structure data of the molecule indicated by the unlabeled data to a user by displaying the structure data on a display device or the like, and may acquire a calculation result input by the user.

The first generation unit 20 generates labeled data by assigning the acquired information on the energy as a correct answer label to the structure data of the molecule of the selected unlabeled data. The first generation unit 20 adds the generated labeled data as energy prediction training data to the energy prediction training data group 30. The first generation unit 20 transfers the generated labeled data to the second generation unit 22.

The second generation unit 22 inputs the structure data of the molecule of the labeled data transferred from the first generation unit 20 to the energy prediction model 24, and acquires a prediction value of the energy. The second generation unit 22 calculates a prediction error from the energy indicated by the correct answer label of the labeled data and the prediction value of the acquired energy.

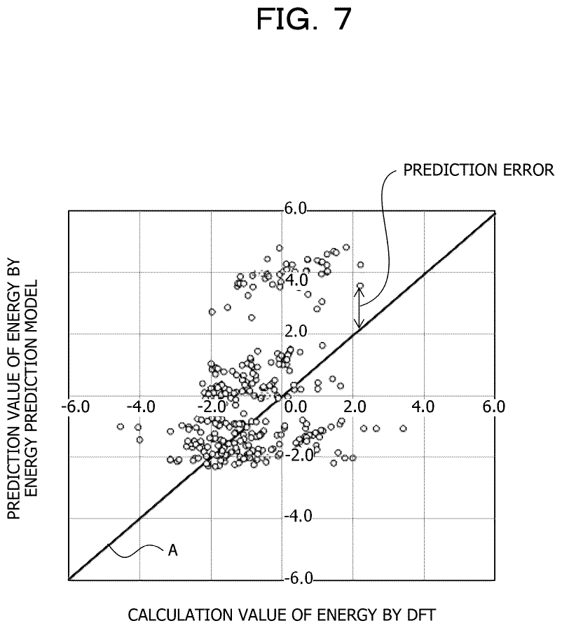

FIG. 7 illustrates an example of a relationship between a calculation value of energy by DFT and a prediction value of the energy by the energy prediction model 24. Each plot of a graph illustrated in FIG. 7 corresponds to a calculation value and a prediction value for one piece of structure data. A line A illustrated in FIG. 7 represents a case where the calculation value and the prediction value coincide with each other, for example, a case where a prediction error is 0. For example, the second generation unit 22 calculates a distance between the line A and each plot as the prediction error for the structure data corresponding to the plot.

The second generation unit 22 generates, as error prediction training data, a set of structure data of a molecule of labeled data transferred from the first generation unit 20 and the calculated prediction error. The second generation unit 22 adds the generated error prediction training data to the error prediction training data group 34.

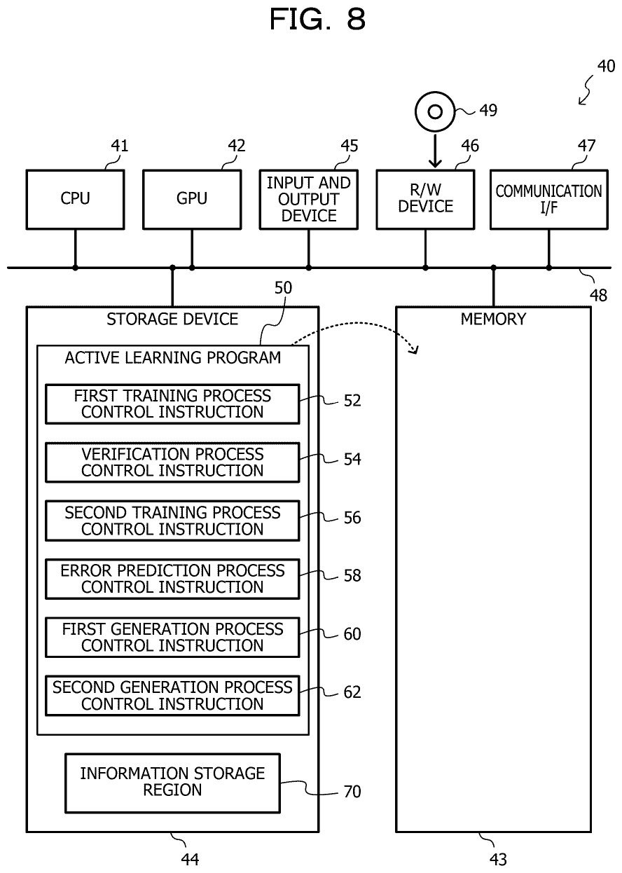

The active learning apparatus 10 may be implemented by a computer 40 illustrated in FIG. 8, for example. The computer 40 includes a central processing unit (CPU) 41, a graphics processing unit (GPU) 42, a memory 43 serving as a temporary storage region, and a non-volatile storage device 44. The computer 40 includes an input and output device 45 such as an input device and a display device, and a read/write (R/W) device 46 that controls reading and writing of data from and to a storage medium 49. The computer 40 includes a communication interface (I/F) 47 that is coupled to a network such as the Internet. The CPU 41, the GPU 42, the memory 43, the storage device 44, the input and output device 45, the R/W device 46, and the communication I/F 47 are coupled to each other via a bus 48.

For example, the storage device 44 is a hard disk drive (HDD), a solid-state drive (SSD), a flash memory, or the like. An active learning program 50 for causing the computer 40 to function as the active learning apparatus 10 is stored in the storage device 44 serving as a storage medium. The active learning program 50 includes a first training process control instruction 52, a verification process control instruction 54, a second training process control instruction 56, an error prediction process control instruction 58, a first generation process control instruction 60, and a second generation process control instruction 62. The storage device 44 includes an information storage region 70 in which information constituting each of the energy prediction model 24 and the error prediction model 26 is stored.

The CPU 41 reads the active learning program 50 from the storage device 44, develops the active learning program 50 into the memory 43, and sequentially executes control instructions included in the active learning program 50. By executing the first training process control instruction 52, the CPU 41 operates as the first training unit 12 illustrated in FIG. 4. By executing the verification process control instruction 54, the CPU 41 operates as the verification unit 14 illustrated in FIG. 4. By executing the second training process control instruction 56, the CPU 41 operates as the second training unit 16 illustrated in FIG. 4. By executing the error prediction process control instruction 58, the CPU 41 operates as the error prediction unit 18 illustrated in FIG. 4. By executing the first generation process control instruction 60, the CPU 41 operates as the first generation unit 20 illustrated in FIG. 4. By executing the second generation process control instruction 62, the CPU 41 operates as the second generation unit 22 illustrated in FIG. 4. The CPU 41 reads information from the information storage region 70, and develops each of the energy prediction model 24 and the error prediction model 26 into the memory 43. Accordingly, the computer 40 that executes the active learning program 50 functions as the active learning apparatus 10. The CPU 41 that executes the programs is hardware. Some of the programs may be executed by the GPU 42.

The functions realized by the active learning program 50 may be realized by, for example, a semiconductor integrated circuit, in more detail, an application-specific integrated circuit (ASIC), a field-programmable gate array (FPGA), or the like.

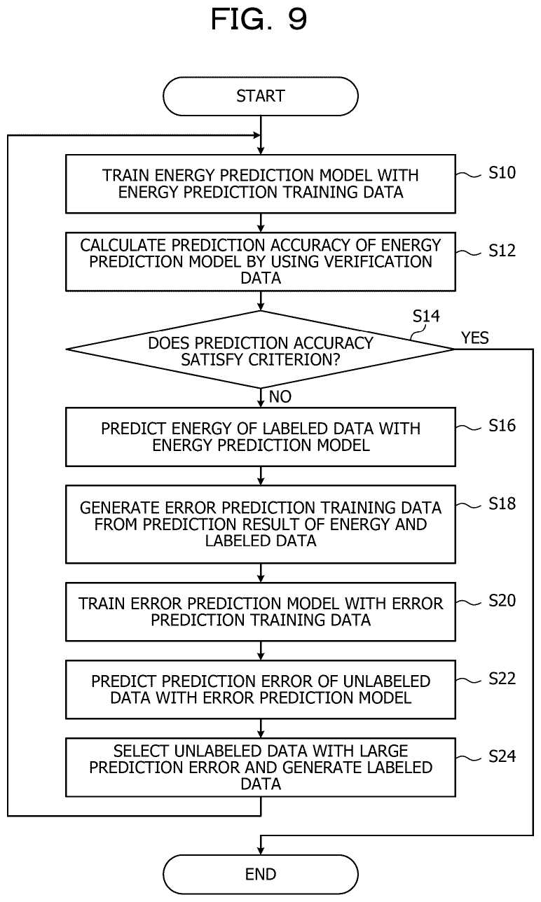

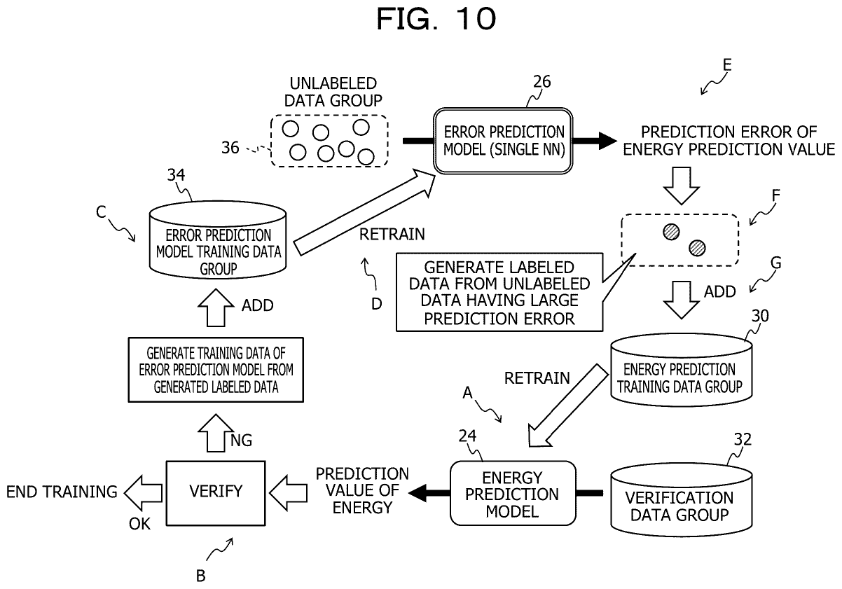

Next, an operation of the active learning apparatus 10 according to the present embodiment will be described. When training of the energy prediction model 24 is instructed, an active learning process illustrated in FIG. 9 is executed in the active learning apparatus 10. The active learning process is an example of an active learning method of the disclosed technique. Hereinafter, the active learning process illustrated in FIG. 9 will be described together with an outline of a process in the active learning apparatus 10 according to the present embodiment illustrated in FIG. 10.

At step S10, the first training unit 12 trains the energy prediction model 24 by using the energy prediction training data group 30 (A in FIG. 10). Next, in step S12, the verification unit 14 calculates prediction accuracy of the energy prediction model 24, based on a prediction value predicted by the energy prediction model 24 for structure data of a molecule of verification data and a correct answer label of the verification data. Next, in step S14, the verification unit 14 determines whether or not the calculated prediction accuracy satisfies a predetermined criterion (B in FIG. 10). In a case where the criterion is not satisfied, the process proceeds to step S16.

At step S16, the second generation unit 22 inputs structure data of a molecule of labeled data to the energy prediction model 24 to acquire a prediction value of energy. Next, in step S18, the second generation unit 22 calculates a prediction error from energy indicated by a correct answer label of the labeled data and the acquired prediction value of the energy. A set of the structure data of the molecule of the labeled data and the calculated prediction error is generated by the second generation unit 22 as error prediction training data, and is added to the error prediction training data group 34 (C in FIG. 10).

Next, in step S20, the second training unit 16 trains the error prediction model 26 by using the error prediction training data group 34 (D in FIG. 10). Next, in step S22, the error prediction unit 18 predicts a prediction error of the energy prediction model 24 for each of a plurality of pieces of unlabeled data included in the unlabeled data group 36, based on the error prediction model 26 (E in FIG. 10).

Next, in step S24, the first generation unit 20 selects unlabeled data with a large predicted prediction error, from the plurality of pieces of unlabeled data. The first generation unit 20 acquires information on energy serving as a correct answer label for the selected unlabeled data, and assigns the acquired information on the energy as the correct answer label to the structure data of the molecule of the selected unlabeled data to generate labeled data (F in FIG. 10). The first generation unit 20 adds the generated labeled data as energy prediction training data to the energy prediction training data group 30 (G in FIG. 10), and the process returns to step S10.

When it is determined that the calculated prediction accuracy satisfies the predetermined criterion in step S14, the active learning process is ended.

As described above, an active learning apparatus according to the present embodiment trains an error prediction model by using, as training data, a set of a plurality of pieces of data and a prediction error of the energy prediction model for each of the plurality of pieces of data. The active learning apparatus predicts a prediction error of the energy prediction model for each of a plurality of pieces of unlabeled data based on the error prediction model, and generates labeled data by assigning a correct answer label to unlabeled data selected from the plurality of pieces of unlabeled data based on the prediction error. By using the generated labeled data, the active learning apparatus retrains the energy prediction model. In this manner, in the present embodiment, unlabeled data having a large prediction error predicted by the error prediction model configured with a single neural network or the like is labeled. Accordingly, it is possible to reduce a training cost by the active learning, as compared with a case where uncertainty of unlabeled data is predicted by a machine learning model configured with a plurality of neural networks.

An experiment result will be described, which verifies that accuracy of a machine learning model may be improved with as few labeled data as possible even in a case where active learning is performed by a method according to the present embodiment (hereafter, referred to as “present method”).

For comparison with the present method in the present verification, a method of freely selecting training data for supervised learning of an energy prediction model, by sampling from prepared training data is used as a comparative method. In the present verification, 3770 pieces of data of sets of structure data of a molecule and energy of the molecule are prepared, 2870 pieces of the data are set as training data, and the remaining 900 pieces of the data are set as verification data not used for training. All pieces of structure data of the 2870 pieces of training data are used as an unlabeled data group to be labeled, and sets of all the pieces of structure data of the 2870 pieces of training data and the energy are used for supervised learning.

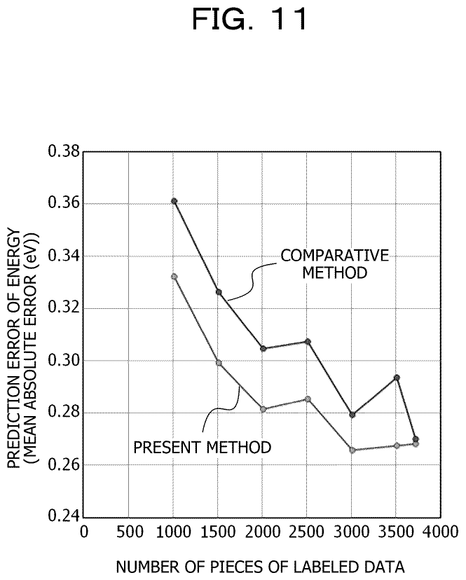

FIG. 11 illustrates, as an experiment result, a relationship between the number of pieces of labeled data used in supervised learning and a prediction error of energy by an energy prediction model with which the supervised learning is executed with the number of pieces of labeled data, for each method. As illustrated in FIG. 11, the present method may improve accuracy of the energy prediction model with a smaller number of labeled data than the comparative method. As a matter of course, in a case where all the labeled data are used for training, the accuracy of the present method is equivalent to accuracy of the comparative method.

Although a case where energy is predicted from structure data of a molecule is described in the embodiment described above, data to which the disclosed technique may be applied is not limited to this example. For example, in a case where it is difficult to prepare a large amount of labeled data, it is highly effective to apply the disclosed technique.

Although the active learning program is stored (installed) in advance in the storage device in the embodiment described above, the embodiment is not limited thereto. The program according to the disclosed technique may be provided in a form of being stored in a storage medium such as a compact disc read-only memory (CD-ROM), a Digital Versatile Disc ROM (DVD-ROM), a Universal Serial Bus (USB) memory, or the like.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an active learning program for causing a computer to execute a process comprising:
predicting, based on a second machine learning model trained by using a set of a plurality of pieces of data and a prediction error of a first machine learning model for each of the plurality of pieces of data as training data, a prediction error of the first machine learning model for each of a plurality of pieces of unlabeled data;
generating labeled data by assigning a correct answer label to unlabeled data selected from the plurality of pieces of unlabeled data, based on the predicted prediction error; and
retraining the first machine learning model by using the generated labeled data.

2. The non-transitory computer-readable recording medium according to claim 1,
wherein unlabeled data with which the predicted prediction error is equal to or more than a predetermined value or a predetermined number of pieces of unlabeled data in descending order of the predicted prediction errors is selected, from among the plurality of pieces of unlabeled data.

3. The non-transitory computer-readable recording medium according to claim 1, the active learning program for causing the computer to execute the process further comprising:
calculating each of the prediction errors from each of prediction results obtained by inputting each of a plurality of pieces of the labeled data to the first machine learning model and each of correct answer labels of the plurality of pieces of labeled data, and generating a plurality of pieces of the training data from each of the calculated prediction errors and each of the plurality of pieces of labeled data; and
training the second machine learning model by using the training data.

4. The non-transitory computer-readable recording medium according to claim 1,
wherein the predicting of the prediction error and the generating of the labeled data are repeated until accuracy of the retrained first machine learning model satisfies a predetermined criterion.

5. The non-transitory computer-readable recording medium according to claim 3,
wherein the second machine learning model is retrained by using the training data generated based on the labeled data generated from the selected unlabeled data.

6. The non-transitory computer-readable recording medium according to claim 1,
wherein the labeled data is data in which information on energy of a molecule is assigned to structure data of the molecule as the correct answer label, and
the first machine learning model is a machine learning model that outputs the energy of the molecule as a prediction result in a case where the structure data of the molecule is input.

7. The non-transitory computer-readable recording medium according to claim 6,
wherein in the generating of the labeled data, the energy of the molecule is calculated from the structure data of the molecule which is the selected unlabeled data, by using density functional theory.

8. An active learning method for causing a computer to execute a process comprising:
predicting, based on a second machine learning model trained by using a set of a plurality of pieces of data and a prediction error of a first machine learning model for each of the plurality of pieces of data as training data, a prediction error of the first machine learning model for each of a plurality of pieces of unlabeled data;
generating labeled data by assigning a correct answer label to unlabeled data selected from the plurality of pieces of unlabeled data, based on the predicted prediction error; and
retraining the first machine learning model by using the generated labeled data.

9. The active learning method according to claim 8,
wherein unlabeled data with which the predicted prediction error is equal to or more than a predetermined value or a predetermined number of pieces of unlabeled data in descending order of the predicted prediction errors is selected, from among the plurality of pieces of unlabeled data.

10. The active learning method according to claim 8, the active learning program for causing the computer to execute the process further comprising:
calculating each of the prediction errors from each of prediction results obtained by inputting each of a plurality of pieces of the labeled data to the first machine learning model and each of correct answer labels of the plurality of pieces of labeled data, and generating a plurality of pieces of the training data from each of the calculated prediction errors and each of the plurality of pieces of labeled data; and
training the second machine learning model by using the training data.

11. The active learning method according to claim 8,
wherein the predicting of the prediction error and the generating of the labeled data are repeated until accuracy of the retrained first machine learning model satisfies a predetermined criterion.

12. The active learning method according to claim 10,
wherein the second machine learning model is retrained by using the training data generated based on the labeled data generated from the selected unlabeled data.

13. The active learning method according to claim 8,
wherein the labeled data is data in which information on energy of a molecule is assigned to structure data of the molecule as the correct answer label, and
the first machine learning model is a machine learning model that outputs the energy of the molecule as a prediction result in a case where the structure data of the molecule is input.

14. The active learning method according to claim 13,
wherein in the generating of the labeled data, the energy of the molecule is calculated from the structure data of the molecule which is the selected unlabeled data, by using density functional theory.

15. An active learning apparatus comprising:
a memory; and
a processor coupled to the memory and configured to:
predict, based on a second machine learning model trained by using a set of a plurality of pieces of data and a prediction error of a first machine learning model for each of the plurality of pieces of data as training data, a prediction error of the first machine learning model for each of a plurality of pieces of unlabeled data;
generate labeled data by assigning a correct answer label to unlabeled data selected from the plurality of pieces of unlabeled data, based on the predicted prediction error; and
retrain the first machine learning model by using the generated labeled data.

16. The active learning apparatus according to claim 15,
wherein unlabeled data with which the predicted prediction error is equal to or more than a predetermined value or a predetermined number of pieces of unlabeled data in descending order of the predicted prediction errors is selected, from among the plurality of pieces of unlabeled data.

17. The active learning apparatus according to claim 15, wherein the processor:
calculates each of the prediction errors from each of prediction results obtained by inputting each of a plurality of pieces of the labeled data to the first machine learning model and each of correct answer labels of the plurality of pieces of labeled data, and generating a plurality of pieces of the training data from each of the calculated prediction errors and each of the plurality of pieces of labeled data; and
trains the second machine learning model by using the training data.

18. The active learning apparatus according to claim 15,
wherein a process to predict the prediction error and a process to generate the labeled data are repeated until accuracy of the retrained first machine learning model satisfies a predetermined criterion.

19. The active learning apparatus according to claim 17,
wherein the second machine learning model is retrained by using the training data generated based on the labeled data generated from the selected unlabeled data.

20. The active learning apparatus according to claim 15,
wherein the labeled data is data in which information on energy of a molecule is assigned to structure data of the molecule as the correct answer label, and
the first machine learning model is a machine learning model that outputs the energy of the molecule as a prediction result in a case where the structure data of the molecule is input.
